(19) United States
(12) Patent Application Publication
(10) Pub. No.: US 2025/0082257 A1
(43) Pub. Date: Mar. 13, 2025

(54) DETECTION AND FORECASTING OF NEUROCOGNITIVE DECLINE USING FUNCTIONAL NEUROIMAGING AND MACHINE LEARNING

(71) Applicant: The Chinese University of Hong Kong, Shatin (CN)

(72) Inventors: Helen Mei Ling MENG, Pak Shek Kok (CN); Patrick Chun Man WONG, Tai Po (CN); Xianmin GONG, Shatin (CN); Yuejiao WANG, Shatin (CN); Helene Hoi Lam FUNG, Shatin (CN); Ka Ho WONG, Shatin (CN)

(73) Assignee: The Chinese University of Hong Kong, Shatin (CN)

(21) Appl. No.: 18/814,269

(22) Filed: Aug. 23, 2024

Related U.S. Application Data
(60) Provisional application No. 63/537,775, filed on Sep. 11, 2023.

(51) Int. Cl. A61B 5/00; G01R 33/48; G01R 33/50; G16H 50/30

(52) U.S. Cl. CPC A61B 5/4088; A61B 5/4064; G01R 33/4806; G01R 33/50; G16H 50/30

(57) ABSTRACT

Predictions of current and/or future neurocognitive decline (NCD) can be based on functional neuroimaging data (e.g., fMRI data) and a machine-learning classifier model. Functional neuroimaging data is obtained while the subject performs a naturalistic language-processing task, such as watching a movie clip, and processed to extract a quantitative feature set. A classifier model is trained using machine learning techniques to predict a subject's NCD status based on the quantitative feature set.

### CROSS-REFERENCE TO RELATED APPLICATION

This application claims the benefit of U.S. Provisional Application No. 63/537,775, filed Sep. 11, 2023, the disclosure of which is incorporated herein by reference.

### BACKGROUND

This disclosure relates generally to assessment of cognitive state and in particular to using functional neuroimaging data from naturalistic language processing tasks and machine learning to detect a current state and/or forecast a future state of neurocognitive decline in a subject.

Neurocognitive decline (NCD) refers generally to conditions of declining cognitive performance beyond normal aging. NCD can be caused by various conditions, including Alzheimer's disease, vascular diseases, etc. Based on the development stage and severity of symptoms, NCD can be categorized as “mild” (m-NCD) or “major” (M-NCD). Mild NCD is also referred to as “mild cognitive impairment,” while major NCD is referred to as “dementia.” It is estimated that 5-8% of the global population aged 60 and above lives with dementia, posing significant public health challenges.

Early detection of NCD is of considerable interest, in part because numerous studies have shown that the progression from mild NCD to major NCD can be slowed, halted, or even reversed, provided that the NCD is detected during early stages.

### SUMMARY

One approach to early detection of NCD involves detecting structural and functional changes in the brain, using neuroimaging techniques such as anatomical magnetic resonance imaging (MRI) and functional MRI (fMRI). It has been theorized that functional changes in the brain may become apparent prior to any structural changes or overt NCD symptoms. However, reliable protocols for detecting and forecasting NCD remain elusive.

Certain embodiments of the present invention relate to protocols for detecting NCD and/or forecasting future development of NCD using functional neuroimaging data (e.g., fMRI data) and a machine-learning classifier model. The functional neuroimaging data is obtained while the subject performs a naturalistic language-processing task, such as watching a movie clip. The movie clip can include realistic scenes involving various characters doing common activities and can include segments of dialog, monolog (e.g., voiceover narration), and non-speech action. The functional neuroimaging data can be processed to extract a quantitative feature set, encompassing features such as brain activation related to natural language processing. A classifier model can be trained (using machine learning techniques) to predict a subject's NCD status based on the quantitative feature set. For instance, supervised learning can be used, with a training pool of subjects who provide functional neuroimaging data and also undergo neurocognitive assessment, which can include completing a cognitive test (such as the Montreal Cognitive Assessment) or receiving a clinical diagnosis; the results of the assessment can be used to assign labels to the training data samples. Depending on whether the neurocognitive assessment is completed concurrently with (e.g., within a few days of) the functional neuroimaging data collection or at a later time (e.g., six months, a year, or more after functional neuroimaging), the resulting NCD prediction can represent an assessment of current NCD status or a forecast of future NCD status.

Some embodiments relate to methods for assessing neurocognitive decline (NCD) in a test subject. Such methods can include: collecting functional neuroimaging data while the test subject performs a naturalistic language-based task; extracting a feature set from the neuroimaging data; defining an input data set that includes at least the feature set; and determining a predicted NCD status for the test subject by analyzing the input data set using a classifier that has been trained using machine learning to predict the NCD status for an individual, wherein training of the classifier is based on corresponding input data sets obtained for a plurality of training subjects having known NCD status based on a neurocognitive assessment. Such methods can be implemented using computer systems, e.g., as program code executed by a process of a computer system.

Some embodiments relate to methods for assessing neurocognitive decline (NCD) in a test subject using one or more types of neuroimaging data, including one or more of: active-state functional neuroimaging data collected while the test subject performs a naturalistic language-based task; resting-state functional neuroimaging data collected while the test subject is at rest; and/or anatomical neuroimaging data characterizing one or more brain structures of the test subject. Such methods can also include extracting a feature set from the functional neuroimaging data; defining an input data set that includes at least the feature set; and determining a predicted NCD status for the test subject by analyzing the input data set using a classifier that has been trained using machine learning to predict the NCD status for an individual, wherein training of the classifier is based on corresponding input data sets obtained for a plurality of training subjects having known NCD status based on a neurocognitive assessment.

In various embodiments, methods can be implemented using program code that can be stored on a computer-readable storage medium and executed by one or more processors in a computing system

The following detailed description, together with the accompanying drawings, will provide a better understanding of the nature and advantages of the claimed invention.

### BRIEF DESCRIPTION OF THE DRAWINGS

FIG. 1 shows a flow diagram of a process for training and using an automated classifier to predict NCD status of an individual subject according to an embodiment of the present invention.

FIG. 2 shows a flow diagram of a process for feature extraction from fMRI data according to some embodiments.

FIG. 3 shows a graphical rendering of a statistical T-value map for a subject, mapped onto a standard Montreal Neurological Institute (MNI) brain template, according to some embodiments.

FIG. 4 shows a graphical rendering of a binary brain mask, mapped onto a standard MNI brain template, according to some embodiments.

FIGS. 5A-5C show histograms of cross-validation results using area under curve (AUC) for trained models according to various embodiments.

FIG. 6 shows a table of average AUC values for trained models according to various embodiments.

FIGS. 7A-7C show histograms of cross-validation results using AUC for trained models according to various embodiments.

FIG. 8 shows a table of average AUC values for trained models according to various embodiments.

FIG. 9 shows a flow diagram of an analysis process that can be used for feature extraction from anatomical MRI data according to some embodiments.

FIGS. 10A-10D show histograms of cross-validation results using AUC for trained models according to various embodiments.

FIG. 11 shows a table of average AUC values for trained models according to various embodiments.

FIGS. 12A-12D show histograms of cross-validation results using AUC for trained models according to various embodiments

FIG. 13 shows a table of average AUC values for trained models according to various embodiments.

### DETAILED DESCRIPTION

The following description of exemplary embodiments of the invention is presented for the purpose of illustration and description. It is not intended to be exhaustive or to limit the claimed invention to the precise form described, and persons skilled in the art will appreciate that many modifications and variations are possible. The embodiments have been chosen and described in order to best explain the principles of the invention and its practical applications to thereby enable others skilled in the art to best make and use the invention in various embodiments and with various modifications as are suited to the particular use contemplated.

According to various embodiments, a subject's neurocognitive decline (NCD) status can be assessed using an automated classifier (e.g., a machine learning model) applied to functional neuroimaging data (e.g., functional magnetic resonance imaging, or fMRI) data obtained while the subject performs a natural-language task such as watching a movie clip. The status can be a binary decision such as “normal” vs. “decline,” or “m-NCD” vs. “M-NCD.” Training of the automated classifier can be based on data from a cohort of subjects from whom comparable fMRI data is obtained and for whom an evaluation of NCD status is obtained using a standard cognitive assessment instrument (such as the Montreal Cognitive Assessment, or MoCA) is determined. In some embodiments, the evaluation is obtained at or near the time of obtaining the fMRI data, and the model is trained to determine (also referred to as predicting, detecting, or classifying) a current NCD status of the subject. In other embodiments, the evaluation is obtained at a later time after obtaining the fMRI data (e.g., a year or two years later), and the model is trained to forecast a future NCD status of the subject.

### NCD Assessment Overview

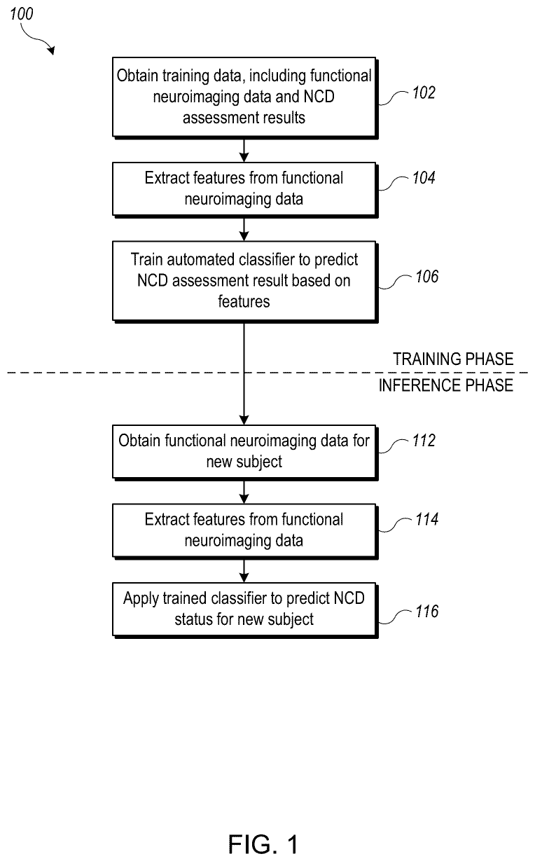

FIG. 1 shows a flow diagram of a process 100 for training and using an automated classifier to predict NCD status of an individual subject according to an embodiment of the present invention. Process 100 can be implemented using a suitably programmed computer system. Blocks 102-106 correspond to a training phase; blocks 112-116 correspond to a testing (or inference) phase.

At block 102, a training data set is obtained. The training data set can include functional neuroimaging data and corresponding NCD assessment results for a number of subjects. The functional neuroimaging data can be obtained using appropriate techniques, such as fMRI, while the subject performs a specific task, such as the movie-watching task described below. Examples of fMRI data collection are described below; however, other neuroimaging modalities can be used. In some embodiments, additional data can be obtained, including demographic data (e.g., patient's age, gender, education level, etc.). NCD assessment results can be obtained by administering a neurocognitive test such as the Montreal Cognitive Assessment (MoCA) or by a clinician's diagnosis. The test results can be used to assign a ground-truth NCD status label to each data point in the training data set. The NCD status label can be a binary label, e.g., either “normal” (NCD not indicated) or “decline” (NCD indicated). In embodiments where the classifier is being trained to predict current NCD status, the neurocognitive test can be administered concurrently with collection of functional neuroimaging data, e.g., on the same day or within a few days. In embodiments where the classifier is being trained to forecast future NCD status (e.g., six months, a year, or two years after the fMRI is performed), the neurocognitive test can be administered at an appropriate point in time after the fMRI data is collected. Training subjects can be selected to reflect a population for which assessing NCD is of interest. For instance, subjects may be adults aged 60 or older (the age range at increased risk for developing NCD), and the distribution of various demographic characteristics (e.g., age, gender, education) can be controlled to provide a representative sample of the population.

At block 104, the functional neuroimaging data can be preprocessed to extract a feature set for input to the automated classifier. For instance, fMRI data can include a sequence of three-dimensional images, providing a large number of voxels and a data set of extremely high dimensionality. To make the machine learning problem more tractable, the data can be analyzed using a multi-step process that identifies brain regions of interest and produces a map for each subject quantifying the activity in each region. Further reduction in feature set size can be obtained, for example, by applying principal component analysis to the maps, or by using a two-step feature selection procedure that includes a Pearson correlation-based feature selection and an L1-penalty feature selection. An example implementation of preprocessing and feature extraction for fMRI images is described below. Other techniques can also be applied.

At block 106, an automated classification algorithm (also referred to as a “classifier”) can be trained using the training data set. Suitable algorithms include machine-learning binary classification algorithms such as a Support Vector Machine (SVM) classifier (also referred to as “SVC”), a Gaussian Naïve Bayes (GNB) classifier, or other classification algorithms. Regardless of the particular algorithm, the classifier can be trained to predict NCD status based on the feature set extracted from the functional neuroimaging data. (As described below, other data can also be provided to supplement the functional neuroimaging data.) In some embodiments, the prediction output from the classifier can be binary (e.g., either “normal” or “decline”). As noted above, the prediction can pertain to the subject's current NCD status or NCD status at a particular time in the future, depending on the time elapsed between obtaining the functional neuroimaging data and performing the neurocognitive testing.

It should be understood that many combinations of classifier algorithm, functional neuroimaging data (and features extracted therefrom), and neurocognitive tests can be used. The neuroimaging data can include fMRI data obtained while performing a naturalistic language-based task (e.g., a movie-watching task as described below), or features extracted from the fMRI data (e.g., as described below). In some embodiments, additional neuroimaging data, such as anatomical MRI data or features extracted from anatomical MRI data, can be provided as input data to the classifier, in addition to functional neuroimaging data. If desired, additional input data can be used, such as demographic data for each subject (e.g., age, gender, education level, etc.). NCD status labels can be assigned based on results of one or more neurocognitive tests, including but not limited to MoCA. In some embodiments, clinicians' diagnoses can be used to assign NCD status labels, and such diagnoses may or may not incorporate results of a specific test.

The trained classifier can thereafter be used to predict NCD status for a new (previously unseen) subject. At block 112, functional neuroimaging data can be obtained for the new subject. The functional neuroimaging data can be of the same kind that was obtained for the training subjects at block 102 and can be obtained using the same protocol (e.g., watching the same movie). At block 114, features can be extracted from the neuroimaging data; the same feature extraction techniques used at block 104 can be applied. At block 116, the trained classifier can be used to predict NCD status for the new subject based on the features extracted at block 114. As noted above, the predicted NCD status can refer to the subject's present-day status (i.e., as of the time the neuroimaging data was obtained) or can be a forecast of future status (e.g., six months, a year, or two years later), depending on how the classifier was trained.

### A Naturalistic Language-Processing Task: Movie-Watching Task

FMRI and other functional neuroimaging techniques can acquire data regarding brain activity while a subject performs a task. In some embodiments, the task is a movie-watching task in which the subject watches a movie clip. To encourage attention, subjects may be told that they will be asked questions about the movie clip after watching it; however, the classifier can be trained and applied without regard to whether any such questions are asked or correctly answered. The movie clip can include multiple segments or scenes showing realistic depictions of daily-life activities. In particular, at least one segment or scene can include two or more characters engaging in conversation (dialog) and/or a speech by a single character or voiceover narrator (monolog), and at least one segment or scene can be a “non-verbal” segment in which one or more characters perform some action(s) but do not speak. All speech can be in the primary language of the subject. A non-verbal segment can include ambient sounds or (instrumental) background music. In some embodiments, the total duration of the movie clip can be approximately 10-15 minutes (e.g., 11 minutes). It should be noted that the presence of these different types of segments allows for modeling of brain activities associated with different cognitive activities (e.g., processing of dialog, monolog, and visual events without speech).

The movie-watching task can provide various advantages compared to other tasks associated with detection of NCD. For instance, the movie clip can present daily-life situations with high ecological validity, as compared to conventional lab-based tasks that may lack real-world relevance (e.g., memorizing an a contextual list of words). As another example, movie-watching can comprehensively cover a number of cognitive domains and activities, including language ability, processing speed, sustained attention and monitoring, memory, executive function, and social cognition. The comprehensive scope can improve sensitivity in detecting various types or degrees of NCD. As yet another example, the movie-watching task is easy to perform and has simple instructions, which may foster engagement and reduce confusion as to what is expected from the subject. In addition, the movie-watching task does not require the subject to speak or move, making it suitable for subjects who may be unable to respond due to non-cognitive disabilities or limitations. Further, because the task simulates a daily activity that most subjects are familiar with (observing, listening to speech), results are less likely to be affected by confounding factors such as cultural background, education, socioeconomic status, or (un)familiarity with the task. Accordingly, the same task can be used with a wider range of populations.

Other natural-language processing tasks can be substituted for the movie-watching task. For instance, the test subject can be presented with a stream of spoken language stimuli, with or without visual accompaniment.

In some embodiments, additional functional neuroimaging data can be collected while the subject is at rest (or in a resting state). The resting state can be a state in which spoken-language stimuli are not provided, and the subject can be awake (e.g., lying or sitting still) or asleep.

### Data Collection and Feature Extraction

In some embodiments, fMRI data can be collected while the subject watches the movie clip. For instance, the subject can be positioned in an fMRI apparatus that allows the subject to view and hear the movie clip while the fMRI apparatus operates to collect images at regular intervals. In various embodiments, the repetition time can be from a few hundred milliseconds to about 2 seconds; in some embodiments, a 900 ms repetition time is used. Images are time-stamped and can be correlated with events in the movie clip (e.g., when speaking occurs).

Various types of fMRI data can be acquired. In some embodiments, T2-weighted blood oxygen-level-dependent (BOLD) images are used. The volume of data generated can be quite large. Accordingly, in some embodiments, feature extraction techniques can be used to extract features of interest from the fMRI data, and the extracted features can be input to the classifier model.

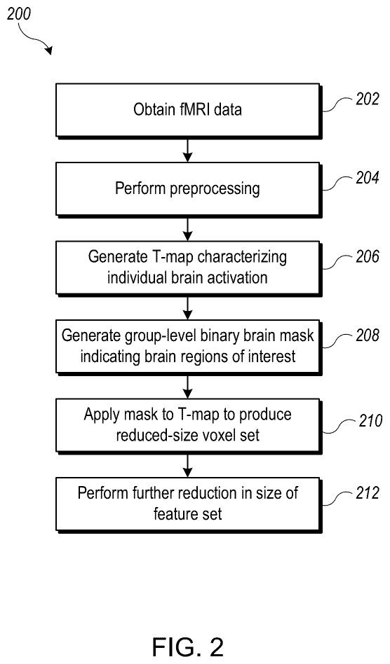

FIG. 2 shows a flow diagram of a process 200 for feature extraction from fMRI data according to some embodiments. Process 200 can begin with obtaining fMRI data at block 202, while the subject is performing the movie-watching task. A variety of fMRI acquisition techniques can be used; specific examples are described below.

At block 204, preprocessing of the fMRI data can be performed. Preprocessing can include various procedures to denoise the data. For example, to ensure magnetization stabilization, the first few images (e.g., 9 BOLD images in one embodiment) can be discarded. Additional preprocessing of the remaining images can be performed using, e.g., the SPM12 (Statistical Parametric Mapping) software package for MATLAB or other established software packages such as AFNI (Analysis of Functional NeuroImages), FSL (FMRIB Software Library), or Nipype. Examples of preprocessing include field map correction, realignment, slice timing correction, co-registration, segmentation, normalization, and spatial smoothing. For example, B0 field map correction can correct for geometric distortion in the signal caused by magnetic field inhomogeneity. Rigid-body realignment can be applied to align images from the same subject. Co-registration, segmentation, and normalization can be performed to map each subject's images to a standard coordinate system such as the Montreal Neurological Institute (MNI) space with affine regularization. Normalized images can be spatially smoothed, e.g., using an isotropic 5-mm full-width-at-half-maximum (FWHM) Gaussian kernel.

At block 206, an individual-level analysis of the (preprocessed) fMRI data can be performed to generate a statistical parametric map for each subject. As used herein, a “statistical parametric map” refers generally to an image map in which the voxel values are, under the null hypothesis, distributed according to a particular probability distribution. In some embodiments, the probability distribution can be the Student's T distribution, and the resulting map is referred to as a “T-map.” In some embodiments, other probability distributions can be substituted for the Student's T distribution, resulting in a different statistical parametric map that can serve similar purposes as the T-map. In some embodiments, the T-map can contain information regarding the association of each voxel with the speech perception function. For instance, using general linear modeling, a subject's fMRI signals can be regressed on: (1) the six rigid-body head motion parameters; (2) a dummy regressor coding the presence of motion spikes, measured by framewise displacement; and (3) two event regressors (presence or absence of speech at the time of acquisition of an image). The rigid-body head motion parameters can be derived, e.g., from the realignment procedure in block 204. A motion spike can be defined as a framewise displacement greater than 1 mm or another criterion (e.g., 0.5 mm or the edge length of a voxel). Motion spikes can be censored by incorporating a dummy/nulling regressor (e.g., 1 for volume with motion spike; 0 for volume without) in the general linear model. Event regressors can be constructed by convolving boxcar reference vectors (capturing onset and duration of particular events in the movie) and a canonical hemodynamic response function without temporal or spatial derivative. A high-pass filter (e.g., with a cutoff period of 128 s) can be used to remove low-frequency signals, and a first-order autoregressive model can be used to control for temporal autocorrelations. For each subject, a voxel-wise univariant contrast is then performed to compute a statistical T value that quantifies each voxel's association with the presence of speech (vs. absence of speech). The T values can be saved as a T-map for each participant, and the T-map can serve as a source of features for the classifier model.

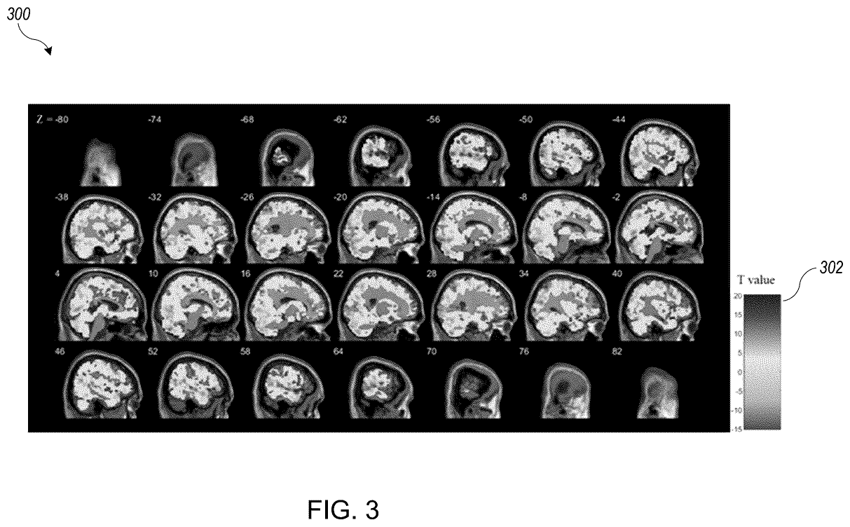

By way of illustration, FIG. 3 shows a graphical rendering of a sagittal view of a T-map 300 for one subject, mapped onto a standard MNI brain template, according to some embodiments. FIG. 3 shows 28 different image slices, with color-coding (using scale 302) to indicate the T value for each voxel in the slice. In this example, a T-value is stored for each voxel of the gray matter, and larger T values correspond to stronger associations between the voxel's activity and language processing (i.e., speech perception for the movie-watching task).

Referring again to FIG. 2, at block 208, a group-level analysis of the statistical parametric maps across subjects can be performed to generate a binary brain mask. The binary brain mask can be used to mark brain regions of interest and reduce the number of voxels subject to feature search and selection. For example, for each subject, a contrast image (showing contrasts between speech and non-speech processing) can be generated from the analysis at block 206. At block 208, the contrast images for a group of subjects can be submitted to a one-sample T-test to identify voxels significantly associated with speech perception. The significance of brain activation can be determined using cluster-extent-based thresholding procedures implemented in SPM12. For instance, a primary voxel-wise threshold of uncorrected p<0.001 (cluster-extent threshold k=0) can be applied to identify suprathreshold voxels. Second, a cluster-level extent threshold, measured by the number of contiguous suprathreshold voxels, can be established based on random field theory (RFT) to identify suprathreshold clusters and control for family-wise error rate (FEW) at the level of P_{FWE}<0.5. The resulting suprathreshold clusters can be saved as a binary brain mask that defines “regions of interest,” from which features will be selected for training of classifier models. The binary bit mask can be defined using a convention that value of “1” is assigned to voxels in a region of interest (e.g., voxels in a suprathreshold cluster) while value of “0” is assigned to other voxels.

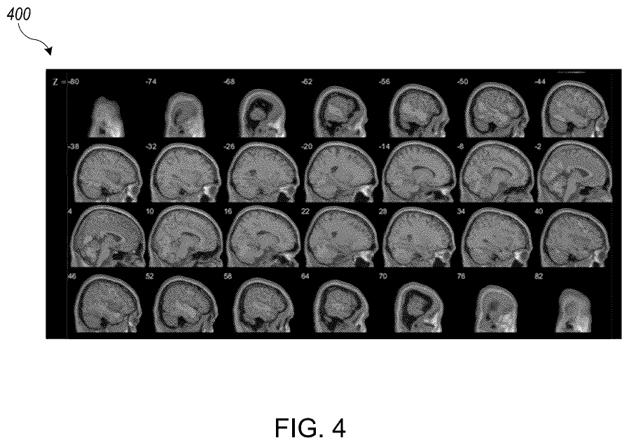

By way of illustration, FIG. 4 shows a graphical rendering of a sagittal view of a binary brain mask 400, mapped onto a standard MNI brain template, according to some embodiments. FIG. 4 shows 28 slices, with color coding to indicate the brain mask. Orange voxels correspond to regions of interest (value 1); other voxels are masked out (value 0).

Referring again to FIG. 2, at block 210, the brain mask generated at block 208 can be applied to the T-maps for each subject to produce a reduced-size voxel set, in which the features are the T-map values for only the voxels that are within regions of interest in the brain mask. As can be seen by comparing FIGS. 3 and 4, a brain mask can significantly reduce the number of voxels of interest. Nevertheless, the reduced-size voxel set may still include a large number of features (e.g., over 15,000). Using such a large feature set as input to a classifier can impose challenges for machine learning due to factors such as information redundancy, difficulty in model convergence (particularly when the number of samples is limited relative to the number of features), and computational resource requirements.

To address these challenges, further reduction in the size of the feature set can be performed. For example at block 212, a statistically-based feature selection procedure can be applied to the masked T maps. In some embodiments, the statistically-based feature selection procedure can be a two-step procedure. The first step can use feature selection based on Pearson correlation, in which only brain voxels with T values that are significantly correlated (e.g., p value<0.01) with the outcome variable (e.g., NCD diagnosis) are kept. The second step can use L1 regularization penalty feature selection to select brain voxels that significantly contribute to predicting the outcome variable. The selected features (voxels) can be used as input data for the machine-learning classifier model. Alternatively, principal component analysis (PCA) can be applied at block 212 to reduce the dimensionality of the reduced-size voxel set. PCA can group correlated features and reduce redundant information. In some embodiments, the reduced-size voxel set produced at block 210 can be decomposed into 10 principal components by using probabilistic algorithms to construct approximate matrix decompositions. (It should be understood that more or fewer than 10 principal components can be used.) Suitable techniques are known in the art. The principal components can be used as input data for the machine-learning classifier model.

### Training and Validation of Classifier

As noted above, a machine-learning classifier can be trained to use input data obtained from fMRI (or other functional neuroimaging modalities) during the movie-watching task to predict an NCD outcome for a subject. In some embodiments, the input data to the classifier can be just the feature set obtained from fMRI data according to process 200. Other input data can be used, including other data derived from fMRI data or other functional neuroimaging data. In some embodiments, the functional neuroimaging data can be supplemented with additional data, such as demographic data and/or structural (or anatomical) neuroimaging data.

The classifier can be trained using supervised learning techniques. For instance, each training subject can complete the movie-watching task, thereby providing fMRI data, and can also complete a standard neurocognitive assessment, such as the MoCA. The standard assessment can provide a ground-truth label for purposes of training: the labels can be binary labels corresponding to “normal” (NCD not indicated) or “decline” (NCD indicated). Training data can be divided into a “training set” (e.g., 75% of the samples) and a “testing set” (e.g., 25% of the samples), with the training samples used to train the models and the testing set used for validation of the model (i.e., assessing performance of the model following training). The training data can be divided using randomized sampling or a stratified shuffle-split method that combines stratified K-fold and shuffle-split to generate stratified randomized folds that preserve the percentage of samples in each class. Other techniques can also be used.

Various machine-learning classifiers can be implemented, including binary classifiers such as an SVM classifier or a GNB classifier. Binary SVM classifiers learn to perform classification by constructing a hyperplane in the input feature space that optimally splits the training samples into the two classes. During training, the SVM learns parameters for an equation defining the hyperplane. Naïve Bayes classifiers perform classification by applying Bayes' theorem, with assumptions of independence between the features. In a Gaussian Naïve Bayes classifier, each continuous feature is assumed to follow a (different) Gaussian distribution within each class, parameters for which are learned from the training samples. For binary classification, Bayes' theorem is used to estimate probability of a sample belonging to each of the two class, given the learned probability distributions, and classification is based on comparing the probabilities for the two classes. Other classifiers can also be implemented.

After training, the testing samples can be used to validate the models by applying the trained classifier to the testing samples and comparing the class predicted by the classifier to the ground-truth class. Based on the number of correct and incorrect results, various metrics can be used to assess performance. In examples described below, the well-known receiver operating characteristic area under curve (AUC) analysis is used as a metric to assess performance of a given classifier. To provide a distribution of results, training and validation procedures can be repeated (using different divisions of the training data each time), thereby providing an AUC distribution. To compare results to chance, a random permutation test can be performed, in which the same testing samples are used but with their ground-truth labels randomly permuted.

It should be understood that models can be trained to predict NCD at different time horizons. For instance, if the standard neurocognitive assessment used to establish ground truth is administered close in time to collection of the functional neuroimaging data (e.g., immediately after, or within a few days), the model's prediction will reflect current (at the time of the functional neuroimaging) NCD status. If the neurocognitive assessment is administered at a later time (e.g., six months later, a year later, two years later, etc.), the model's prediction will reflect a forecast of future NCD status.

To illustrate the effectiveness of this approach, specific example implementations and results will now be described. It should be understood that embodiments of the invention are not limited to these examples.

### Example 1

In one implementation, fMRI data collected during a movie-watching task as described above was used to assess current NCD status of subjects.

Ninety-seven older adult subjects (ages 60-87 years, with a mean of 71.45 and standard deviation of 6.01) underwent fMRI while performing the movie-watching task. Shortly after completing the movie-watching task, each subject completed the MoCA. Based on the MoCA result, each subject's ground-truth NCD status was defined as “normal” or “decline.” Additional demographic data was also collected, including gender (43 female, 54 male) and educational level (mean 7.97 years, standard deviation 3.84 years).

FMRI data were acquired using a Siemens MAGNETOM Prisma 3 Tesla MRI Scanner with a 64-channel head/neck coil. A multiband (factor=6) gradient echo echoplanar (EPI) sequence was used to scan the whole brain to obtain T2-weighted blood oxygen-level-dependent (BOLD) images, with the following scanning parameters: repetition time (TR)=900 ms; echo time (TE)=24 ms; flip angle=90°; voxel size=2×2×2 mm³; matrix size=104×104; field of view (FoV)=206×206 mm²; number of slices=72 (interleaved, transversal, and co-planar to the anterior/posterior commissure plane). Before the BOLD scan, a B0 field map was acquired using a double-echo gradient recalled echo sequence in the same orientation and FoV, with TR=530 ms; short TE=4.92 ms; long TE=7.38 ms; flip angle=60°; voxel size=3×3×3 mm³; number of slices=50. Magnitude and phase images were reconstructed. FMRI image data was processed according to process 200 to obtain an fMRI feature set for input to a classifier model.

To assess the effectiveness of the fMRI feature set as a predictor for subjects' concurrent NCD status (measured in the same time period as the fMRI data acquisition), six training and validation studies were performed. GNB and SVM (also referred to as “SVC”) classifiers were separately trained and validated 1000 times (with different divisions of the training data into training and testing sets), using each of the following sets of input data: (1) only demographic data; (2) only the fMRI feature set; and (3) both the fMRI feature set and the demographic data. In addition, to provide an AUC estimate corresponding to random chance, each trained classifier was validated using random permutation of the validation labels.

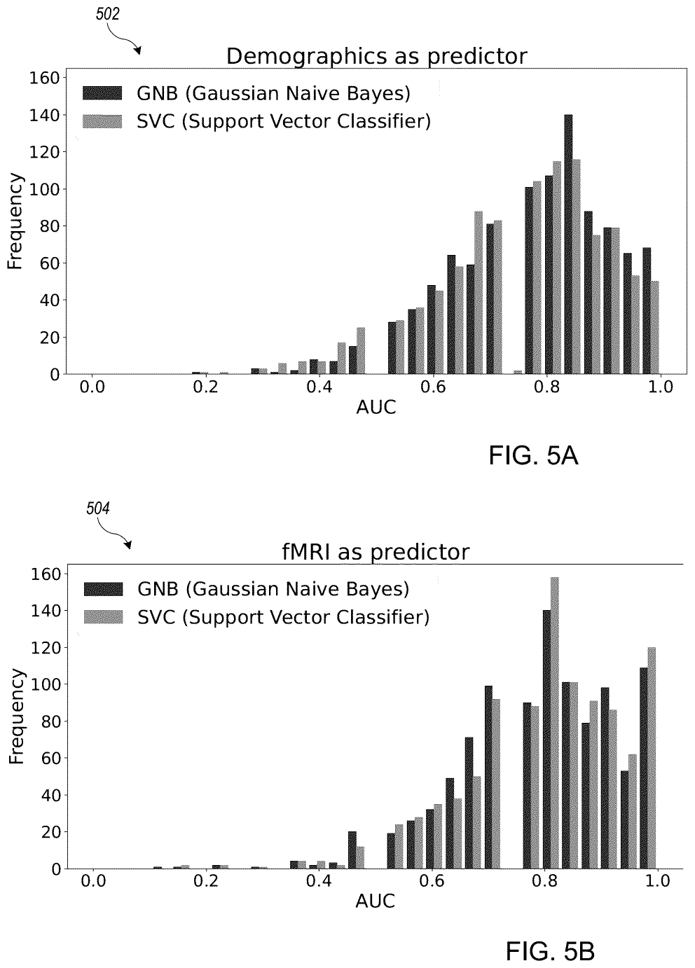

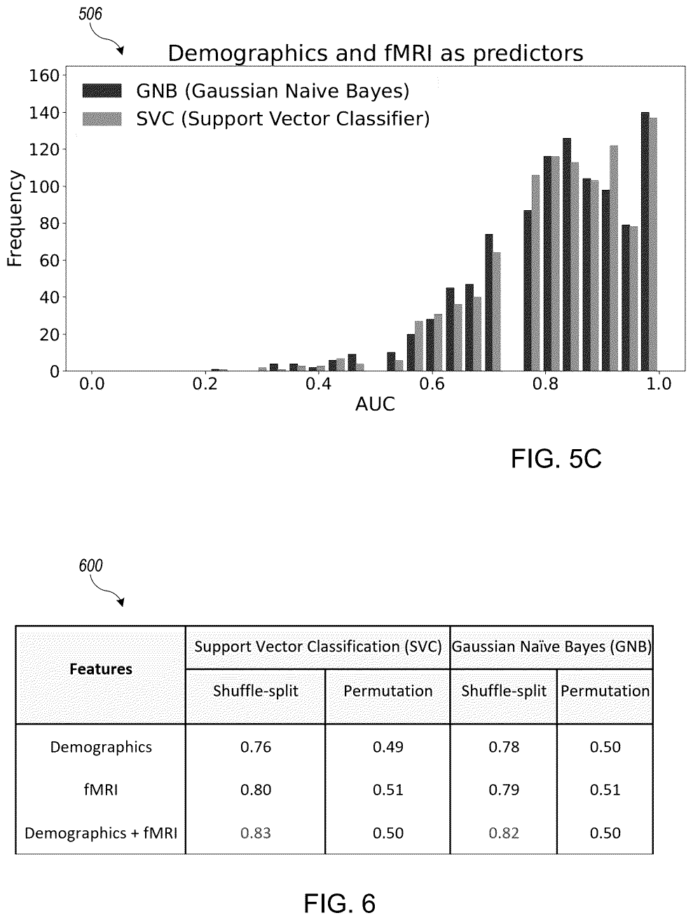

FIGS. 5A-5C show histograms of the AUC results obtained by applying the trained models to the testing data sets. In FIG. 5A, histogram 502 shows results for GNB and SVM classifiers using only demographic data. In FIG. 5B, histogram 504 shows results for GNB and SVM classifiers using only fMRI feature set. In FIG. 5C, histogram 506 shows results for GNB and SVM classifiers using both fMRI feature set and demographic data. Further summarizing the results, FIG. 6 shows a table 600 of average AUC values for the different models validated using stratified shuffle-split and random permutation.

As FIGS. 5A-5C and 6 indicate, the classifiers based on fMRI features were effective for classifying subjects' concurrent NCD status. Either classifier performed better when trained with the fMRI feature set than with demographic data (p<0.001), and both classifiers outperformed random permutation (p<0.001).

### Example 2

In another implementation, fMRI data collected during a movie-watching task was used to forecast NCD status of subjects approximately two years after the fMRI data collection.

To obtain ground-truth NCD status a year after the fMRI data, 50 subjects from the cohort of Example 1 completed the MoCA a second time, two years after the fMRI data was obtained. In this example, two MoCA scores were available for each subject: a “baseline” score obtained at the time of the fMRI data and a ground-truth score obtained two years later.

Six training and validation studies were performed. GNB and SVM classifiers were separately trained and validated 1000 times (with different divisions of the training data into training and testing sets), using each of the following sets of input data: (1) only the demographic data; (2) only the fMRI feature set; and (3) the demographic data and the fMRI feature set. In addition, to provide an AUC estimate corresponding to random chance, each trained classifier was validated using random permutation of the validation labels.

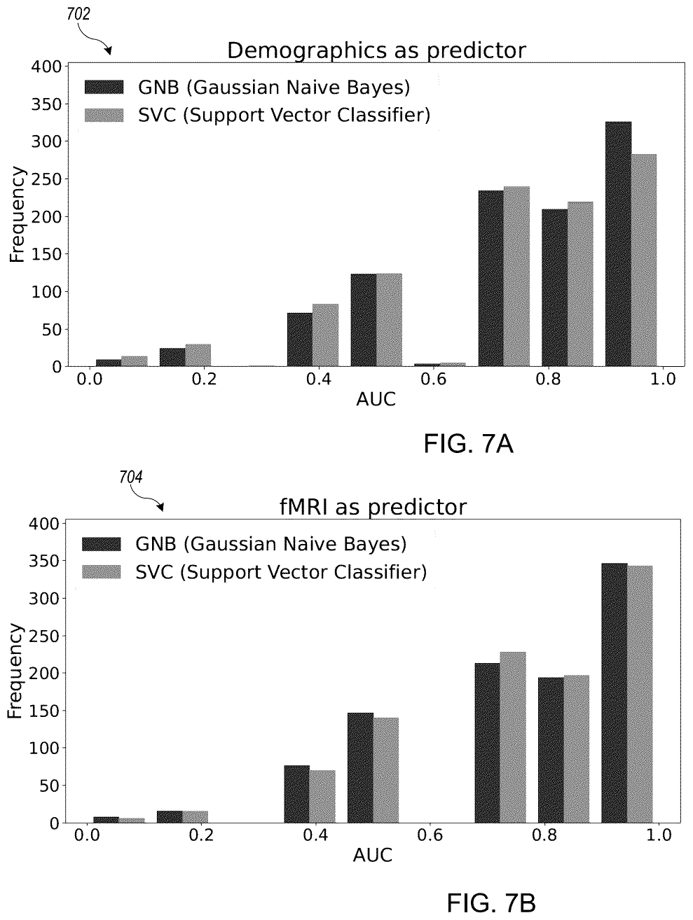

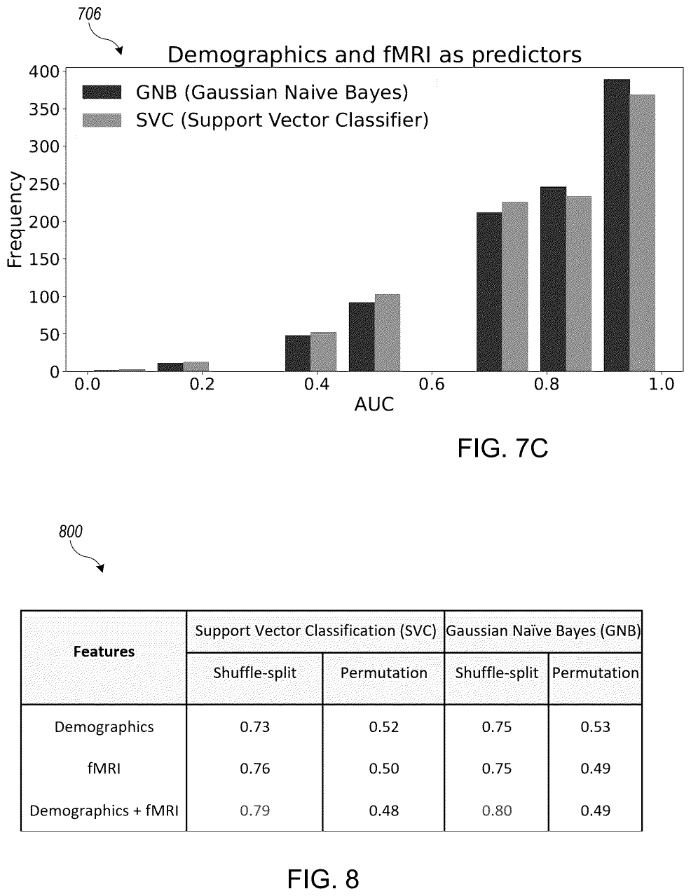

FIGS. 7A-7C show histograms of the AUC results obtained by applying the trained models to the testing data sets. In FIG. 7A, histogram 702 shows results for GNB and SVM classifiers using only demographic data. In FIG. 7B, histogram 704 shows results for GNB and SVM classifiers using only fMRI feature set. In FIG. 7C, histogram 706 shows results for GNB and SVM classifiers using fMRI feature set and demographic data. Further summarizing the results, FIG. 8 shows a table 800 of average AUC values for the different models validated using stratified shuffle-split and random permutation.

As FIGS. 7A-7C and 8 indicate, the classifiers based on fMRI feature sets were effective for forecasting subjects' two-year follow-up NCD status. Models that included the fMRI feature set performed better than models based on demographic data (p<0.001) and models based on random permutation (p<0.001).

### Example 3

The foregoing examples rely on fMRI data, which provides information related to brain function. In research and clinical practice, brain structure is often used, either independently or in combination with other assessments (e.g., neurocognitive tests, blood assays) to aid in NCD detection and diagnosis. Such information can be obtained using structural MRI (sMRI) techniques, also sometimes referred to as “anatomical MRI.” In some embodiments, anatomical information, including sMRI features and/or other information related to brain structure, can also be included in the model and may further improve performance or robustness.

To assess the effect of including anatomical MRI data as input to the classifier, and to compare the effectiveness of anatomical and functional MRI data, sagittal T1-weighted high resolution structural images were also acquired from the subjects in Example 1. Image acquisition used a magnetization-prepared rapid gradient echo (MPRAGE) sequence, with the following scanning parameters: TR=1800 ms; TE=2.53 ms; flip angle=8°; voxel size=0.8×0.8×0.8 mm³; matrix size=272×272; FoV=240×240 mm²; number of slices=208 (sagittal).

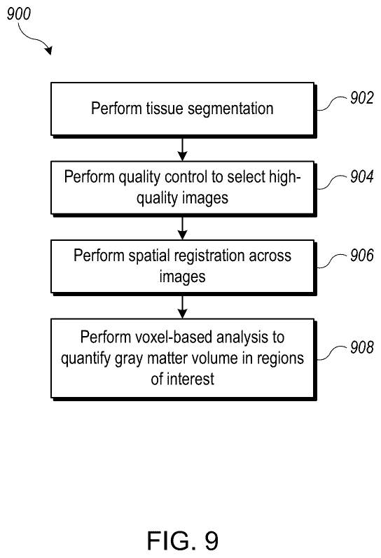

To extract a feature set from the sMRI data, voxel-based morphometry (VBM) analysis was conducted on each subject's T1-weighted brain image to quantify the volumes of gray matter in different brain regions. The analysis was performed using the standard pipeline implemented in the CAT12 (based on SPM12) software package. (Other pipelines can be substituted). FIG. 9 shows a flow diagram of an analysis process 900 that can be used to extract a feature set from sMRI data according to some embodiments. Process 900 includes tissue segmentation, spatial registration, quality check, and region of interest (ROI) analysis.

At block 902, each T1-weighted brain image can be segmented into distinct tissue types (e.g., gray matter, white matter, cerebrospinal fluid, bone, soft tissue, and background), using an automated segmentation process. At block 904, quality control can be performed to assess the quality of segmentation. For instance, the segmented images can be visually inspected, and a set of objective metrics, including noise contrast ratio (NCR), inhomogeneity contrast ratio (ICR), and root-mean-square (RMS) resolution, can be computed and integrated into a weighted average image quality rating (IQR). At block 906, the segmented images that passed the quality control at block 904 can be co-registered to the mean T1-weighted image of all subjects and subsequently normalized to a standard coordinate system such as the MNI space. At block 908, after spatial registration, brain atlases (such as AAL3, the Local-Global Intrinsic Functional Connectivity Parcellation by Schaefer, or the HCP Multi-Modal Parcellation) can be employed to label and quantify the gray matter volumes in various regions of interest (ROIs), where the ROIs are selected to include regions associated with language processing (or other brain functions related to NCD, such as attention and memory). The volumes of gray matter, white matter, and cerebrospinal fluid in the ROIs can be used as an sMRI feature set for training a classifier. Total intracranial volume (TIV) can be calculated and controlled as a covariate, in order to correct for different brain sizes and volumes.

Volume data obtained from 97 subjects' T1 images for selected ROIs was used in model training and cross-validation procedures similar to those described above with reference to Example 1 to classify subjects' current NCD status, in various combinations with the fMRI feature sets and demographic data. Fourteen training and validation studies were performed. GNB and SVM classifiers were each trained and validated 1000 times (with different divisions of the training data into training and testing sets), using each of the following sets of input data: (1) only demographic data; (2) only the fMRI feature set; (3) only the sMRI data; (4) the demographic data and the fMRI feature set; (5) the demographic data and the sMRI data; (6) the fMRI feature set and the sMRI data; and (7) the demographic data, the fMRI feature set, and the sMRI data. Each trained classifier was validated by comparing results with chance-level performance obtained using random permutation.

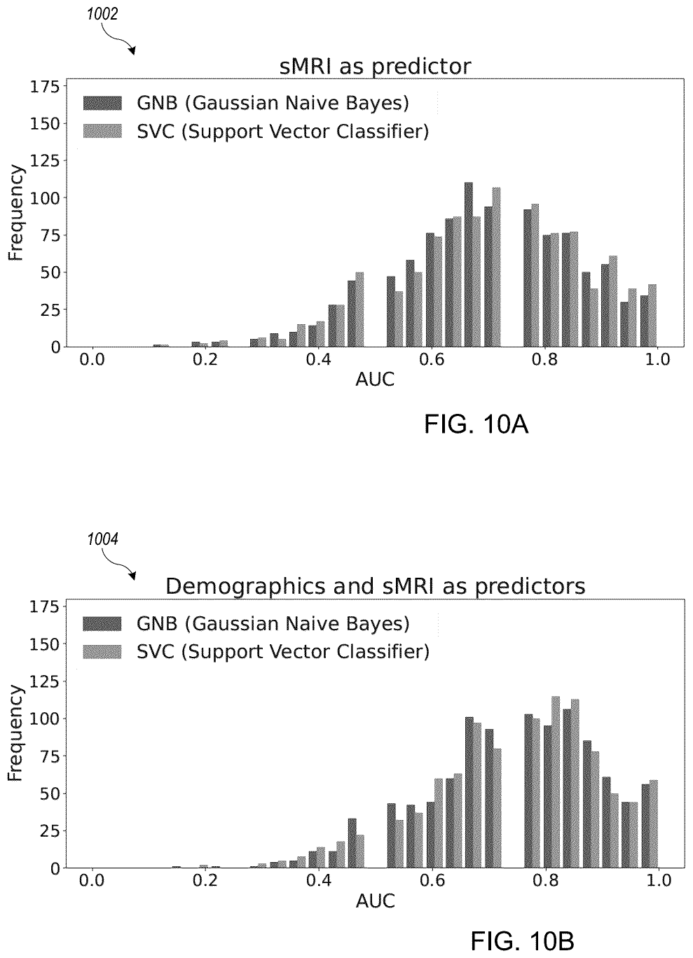

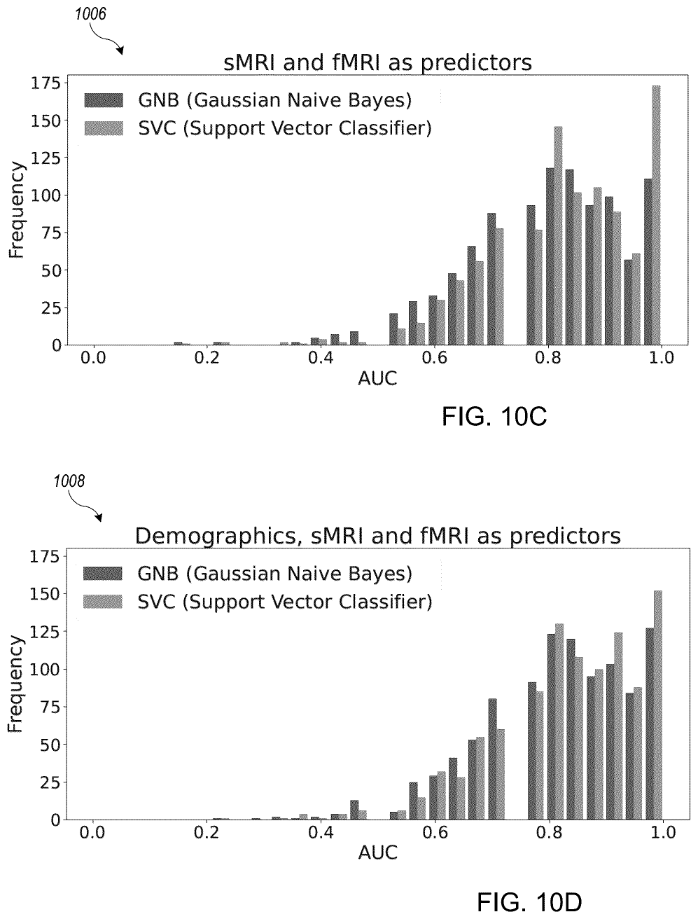

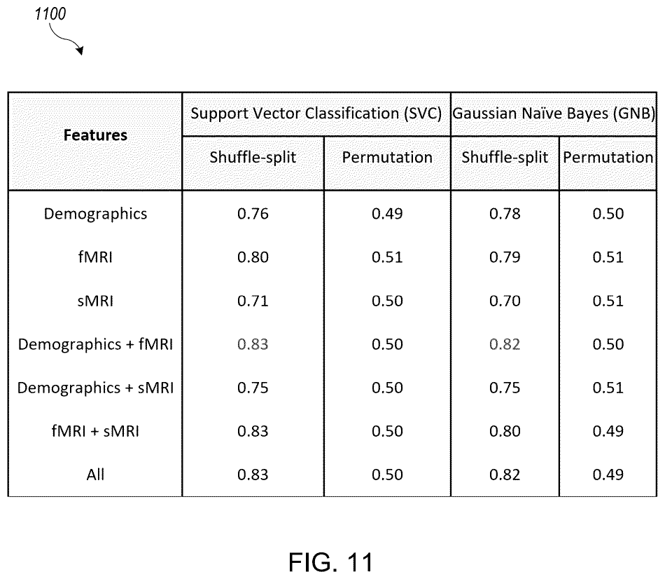

FIGS. 10A-10D show histograms of the AUC results obtained by applying the trained models to the testing data sets. In FIG. 10A, histogram 1002 shows results for GNB and SVM classifiers using only sMRI data. In FIG. 10B, histogram 1004 shows results for GNB and SVM classifiers using demographic and sMRI data. In FIG. 10C, histogram 1006 shows results for GNB and SVM classifiers using fMRI and sMRI data. In FIG. 10D, histogram 1008 shows results for GNB and SVM classifiers using demographic, fMRI, and sMRI data. FIG. 11 shows a table 1100 of average AUC values for the different models validated using stratified shuffle-split and random permutation.

As FIGS. 10 and 11 indicate, the AUC of classifiers based on sMRI data alone was higher than the chance-level but lower than the AUC of classifiers based on fMRI feature sets (p<0.001), and including sMRI data together with the fMRI feature set had negligible effect compared to classifiers using just the fMRI feature set. This example shows that functional neuroimaging data (such as fMRI data) can provide a more powerful tool than structural data (such as sMRI data) for predicting subjects' current NCD status. This result is consistent with other studies suggesting that functional brain changes may precede structural brain changes during the progression of NCD.

### Example 4

In another implementation, fMRI data collected during a movie-watching task and structural MRI (sMRI) data were used to forecast subjects' NCD status approximately two years after the fMRI and sMRI data collection. Collection and preprocessing of fMRI and sMRI data was performed similarly to the examples described above. The subjects were the same as described in Example 2.

Fourteen training and validation studies were performed. GNB and SVM classifiers were each trained and validated 1000 times (with different divisions of the training data into training and testing sets), using each of the following sets of input data: (1) only demographic data; (2) only the fMRI feature set; (3) only sMRI data; (4) the demographic data and the fMRI feature set; (5) the demographic data and the sMRI data; (6) the fMRI feature set and the sMRI data; and (7) the demographic data, the fMRI feature set, and the sMRI feature set. Each trained classifier was validated by comparing with the chance-level performance obtained using random permutation.

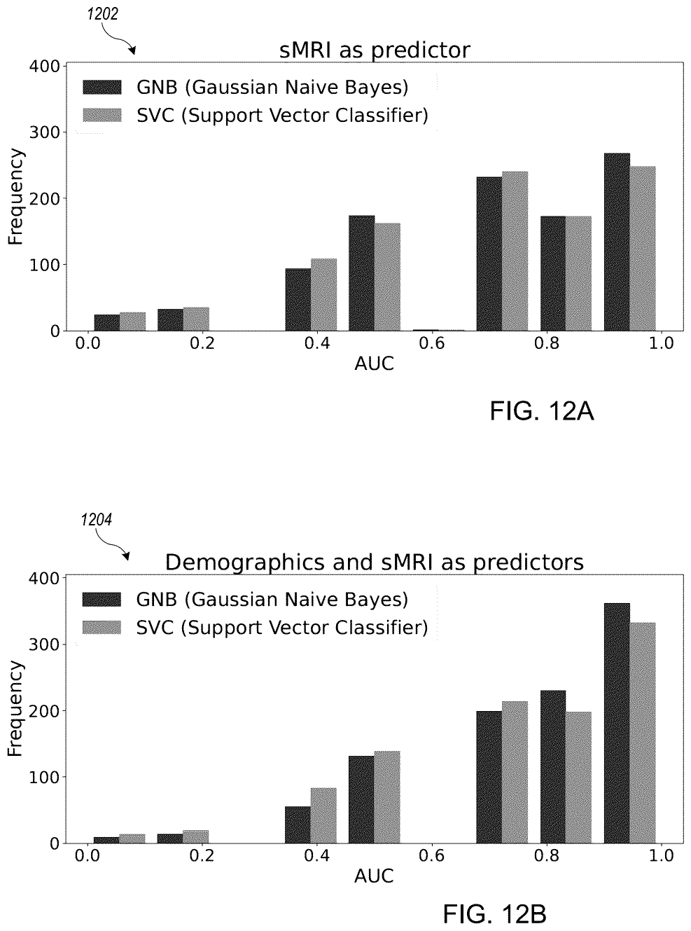

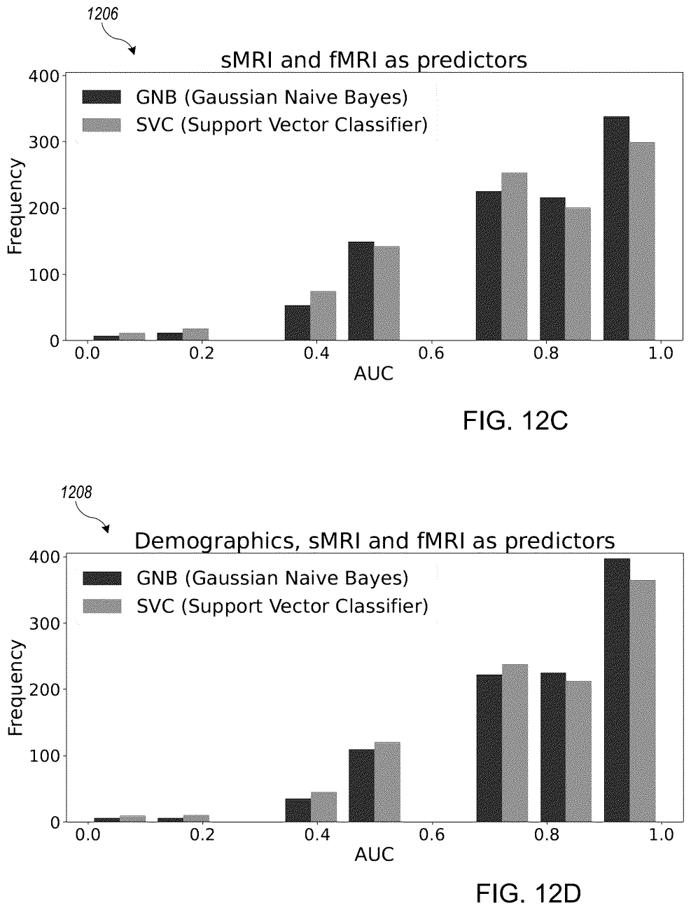

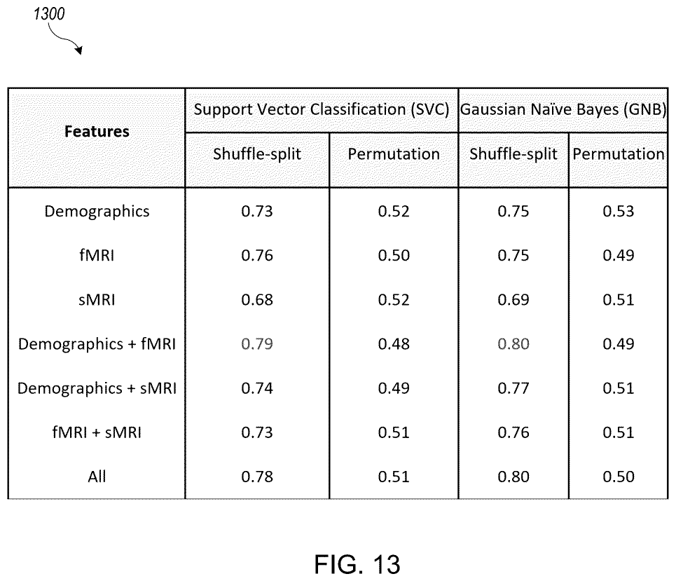

FIGS. 12A-12D show histograms of the AUC results obtained by applying the trained models to the testing data sets. In FIG. 12A, histogram 1202 shows results for GNB and SVM classifiers using only sMRI data. In FIG. 12B, histogram 1204 shows results for GNB and SVM classifiers using demographic and sMRI data. In FIG. 12C, histogram 1206 shows results for GNB and SVM classifiers using fMRI and sMRI data. In FIG. 12D, histogram 1208 shows results for GNB and SVM classifiers using demographic, fMRI, and sMRI data. FIG. 13 shows a table 1300 of average AUC values for the different models validated using stratified shuffle-split and random permutation.

As FIGS. 12 and 13 indicate, the AUC of classifiers based on sMRI data alone was higher than the chance-level but lower than the AUC of classifiers based on fMRI feature sets (p<0.001), and including sMRI data together with the fMRI feature set had negligible effect compared to classifiers using just the fMRI feature set. This example shows that functional neuroimaging data (such as fMRI data) can provide a more powerful tool than structural data (such as sMRI data) for predicting subjects' future NCD status. This result is consistent with other studies suggesting that functional brain changes may precede structural brain changes during the progression of NCD.

### Computer Implementations

Data analysis and computational operations of the kind described herein can be implemented in computer systems that may be of generally conventional design. Such systems may include one or more processors to execute program code (e.g., general-purpose microprocessors usable as a central processing unit (CPU) and/or special-purpose processors such as graphics processors (GPUs) or neural processing units (NPUs) that may provide enhanced parallel-processing capability; memory and other storage devices to store program code and data; user input devices (e.g., keyboards, pointing devices such as a mouse or touchpad, microphones); user output devices (e.g., display devices, speakers, printers); combined input/output devices (e.g., touchscreen displays); signal input/output ports; network communication interfaces (e.g., wired network interfaces such as Ethernet interfaces and/or wireless network communication interfaces such as Wi-Fi); and so on. Various processors or microprocessors can be configured to perform operations described herein by providing suitable program code. Building and testing of classifiers as described herein (including SVM and/or GNB classifiers) can be supported using existing application software such as MATLAB, Python, or custom-built application software. Such software may be said to configure the processor to perform various operations, including operations described herein.

Computer programs incorporating various features of the present invention may be encoded and stored on various computer readable storage media; suitable media include magnetic disk or tape, optical storage media such as compact disk (CD) or DVD (digital versatile disk), flash memory, and other non-transitory media. (It is understood that “storage” of data is distinct from propagation of data using transitory media such as carrier waves.) Computer readable media encoded with the program code may be packaged with a compatible computer system or other electronic device, or the program code may be provided separately from electronic devices (e.g., via Internet download or as a separately packaged computer-readable storage medium).

In alternative embodiments, a purpose-built processor may be used to perform some or all of the operations described herein. Such processors may be optimized, e.g., for performing computations associated with a particular classifier algorithm, and may be incorporated into computer systems of otherwise conventional design or other computer systems. Dedicated or fixed-function circuits can be configured to perform operations by providing a suitable arrangement of circuit components (e.g., logic gates, registers, switches, etc.); automated design tools can be used to generate appropriate arrangements of circuit components implementing operations described herein. Various blocks might or might not be reconfigurable depending on how the initial configuration is obtained. Embodiments of the present invention can be realized in a variety of apparatus including electronic devices implemented using a combination of circuitry and software.

### Additional Embodiments

As described above, embodiments of the invention provide machine-learning techniques that can be used to train an automated classifier to assess current NCD status or forecast future NCD status of a subject based on functional neuroimaging data, alone or in combination with other data such as anatomical (or structural neuroimaging data) and/or demographic data (e.g., age, gender, educational level). The assessed NCD status can be used to inform decisions regarding monitoring and/or treatment of a subject's condition.

While the invention has been described with reference to specific embodiments, those skilled in the art will appreciate that variations and modifications are possible. For instance, all processes described herein are also illustrative and can be modified. Operations can be performed in a different order from that described, to the extent that logic permits; operations described above may be omitted or combined; and operations not expressly described above may be added.

Neuroimaging data characterizing neurological function and/or structure may be collected using a number of techniques. For functional data, techniques include but are not limited to fMRI. For structural data, techniques include but are not limited to anatomical MRI. Other imaging techniques may be used to produce neurological data characterizing brain composition or function. For instance, functional near-infrared spectroscopy (fNIRS), magnetoencephalography (MEG), and/or other neuroimaging modalities can be used to collect functional neuroimaging data. In some embodiments, data obtained using multiple modalities can be combined.

For obtaining training data, NCD can be assessed using standard assessment tests such as MoCA, the Mini-Mental State Exam (MMSE), or other tests. Such tests generally include questions or activity prompts (e.g., drawing a clock or memorizing a short list of words) to assess various aspects of cognitive function, such as memory, attention, executive function, language, object recognition, reasoning, etc. Existing or other tests can be used. Depending on the particular assessment test, the score may be binary (e.g., normal or decline) or may provide a rating indicating severity of NCD. In some embodiments, clinicians' diagnoses (which may or may not be informed by results of a particular test) can be used in place of standard assessment tests.

Training of models can be tailored to specific subpopulations, e.g., a particular age range, gender, educational level, or the like.

In some embodiments, longitudinal tracking can be implemented, in which a classifier predicts current and future NCD status for a succession of time horizons (e.g., now, six months from now, a year from now, etc.). This can be accomplished by training a model for each desired time horizon.

A variety of classifiers (machine-learned algorithms that can be trained to predict an outcome for an unseen data sample based on a set of data samples with known outcomes) can be used. In some instances, a linear or non-linear SVM or GNB classifier provides an effective binary classifier that can be used to indicate presence or absence of NCD. Further, while the foregoing description focuses on binary classifiers, other embodiments may provide other predictions. For instance, if an assessment test provides a score that quantifies likelihood or severity of NCD (e.g., as a continuous variable or other multi-valued variable), such scores can be used to train a machine learning model to predict likelihood or severity of NCD. Suitable models include RankSVM and Support Vector Regression (SVR). Other classifiers can be based on other statistical techniques, such as random forest methods, Bayesian inferencing, or univariate or multivariate analytics. Still other algorithms, such as Hidden Markov Model, and deep learning algorithms (e.g., artificial neural networks) may be substituted. Those skilled in the art with the benefit of this disclosure will be able to implement additional embodiments using these and other classifiers. The parameters used for training and testing the classifier may be varied, including the size of training data sets and the particular combination of inputs. A particular algorithmic implementation of training is not required.

NCD assessments generated in the manner described herein may be used to inform decisions regarding treatment, including interventions aimed at slowing or reversing NCD. Assessments of NCD status may also be used to inform decisions regarding ongoing monitoring, assistance with daily activities, or the like.

Accordingly, although the invention has been described with respect to specific embodiments, it will be appreciated that the invention is intended to cover all modifications and equivalents within the scope of the following claims.

What is claimed is:

1. A method for assessing neurocognitive decline (NCD) in a test subject, the method comprising:
collecting neuroimaging data from the test subject, the neuroimaging data including one or more of:
active-state functional neuroimaging data collected while the test subject performs a naturalistic language-based task;
resting-state functional neuroimaging data collected while the test subject is at rest; or
anatomical neuroimaging data characterizing one or more brain structures of the test subject;
extracting a feature set from the functional neuroimaging data;
defining an input data set that includes at least the feature set; and
determining a predicted NCD status for the test subject by analyzing the input data set using a classifier that has been trained using machine learning to predict the NCD status for an individual, wherein training of the classifier is based on corresponding input data sets obtained for a plurality of training subjects having known NCD status based on a neurocognitive assessment.

2. The method of claim 1 wherein the predicted NCD status is one of:
a binary status that distinguishes between a normal neurocognitive status and presence of NCD;
a continuous variable that represents severity of NCD; or
a continuous variable that represents likelihood of NCD.

3. The method of claim 1 wherein the predicted NCD status corresponds to a present condition of the test subject.

4. The method of claim 1 wherein the predicted NCD status corresponds to a forecast of a future condition of the test subject.

5. The method of claim 1 wherein the neurocognitive assessment for the training subjects is determined using one or both of a standard cognitive assessment test or a clinician's diagnosis.

6. The method of claim 1 wherein the neuroimaging data includes the active-state functional neuroimaging data and the active-state functional neuroimaging data is obtained using one or more of:
functional magnetic resonance imaging (fMRI) data;
functional near-infrared spectroscopy (fNIRS) data; or
magnetoencephalography (MEG) data.

7. The method of claim 6 wherein the naturalistic language-based task includes one or more of:
a movie-watching task in which the test subject is shown a movie clip that includes at least one segment with dialog or monolog and at least one non-speaking segment; or
a listening task in which the test subject is presented with a stream of spoken-language stimuli.

8. The method of claim 1 wherein the neuroimaging data includes a sequence of images of the test subject's brain, each image comprising a plurality of voxels, and wherein extracting the feature set from the neuroimaging data includes:
generating, based on the sequence of images, a T-map characterizing activation of voxels in response to stimulus;
applying a binary brain mask to select a plurality of regions of interest; and
applying a statistically-based feature selection procedure to the voxels of the T-map that are in the regions of interest, wherein the statistically-based feature selection procedure includes one or more of feature selection based on Pearson correlation, L1 regularization penalty feature selection, or principal component analysis.

9. The method of claim 8 wherein the binary brain mask is defined based on a group-level analysis of respective T-maps generated for the training subjects.

10. The method of claim 1 wherein the classifier is one of:
a support vector machine classifier;
a Bayesian classifier;
a Gaussian Naïve Bayes classifier; or
a random forest classifier.

11. The method of claim 1 further comprising:
obtaining demographic data for the test subject,
wherein the input data set further includes the demographic data.

12. The method of claim 1 wherein the input data set includes the active-state functional neuroimaging data and the anatomical neuroimaging data.

13. A system comprising:
a memory; and
a processor coupled to the memory and configured to:
obtain neuroimaging data from a test subject, the neuroimaging data including active-state functional neuroimaging data collected while the test subject performs a naturalistic language-based task;
extract a feature set from the functional neuroimaging data;
define an input data set that includes at least the feature set; and
determine a predicted NCD status for the test subject by analyzing the input data set using a classifier that has been trained using machine learning to predict the NCD status for an individual, wherein training of the classifier is based on corresponding input data sets obtained for a plurality of training subjects having known NCD status based on a neurocognitive assessment.

14. The system of claim 13 wherein the predicted NCD status is one of:
a binary status that distinguishes between a normal neurocognitive status and presence of NCD;
a continuous variable that represents severity of NCD; or
a continuous variable that represents likelihood of NCD.

15. The system of claim 13 wherein the predicted NCD status corresponds to a present condition of the test subject.

16. The system of claim 13 wherein the predicted NCD status corresponds to a forecast of a future condition of the test subject.

17. The system of claim 13 wherein the neurocognitive assessment for the training subjects is determined using one or both of a standard cognitive assessment test or a clinician's diagnosis.

18. The system of claim 13 wherein the neuroimaging data includes the active-state functional neuroimaging data is obtained using one or more of:
functional magnetic resonance imaging (fMRI) data;
functional near-infrared spectroscopy (fNIRS) data; or
magnetoencephalography (MEG) data.

19. The system of claim 18 wherein the naturalistic language-based task includes one or more of:
a movie-watching task in which the test subject is shown a movie clip that includes at least one segment with dialog or monolog and at least one non-speaking segment; or
a listening task in which the test subject is presented with a stream of spoken-language stimuli.

20. The system of claim 13 wherein the neuroimaging data includes a sequence of images of the test subject's brain, each image comprising a plurality of voxels, and wherein extracting the feature set from the neuroimaging data includes:
generating, based on the sequence of images, a T-map characterizing activation of voxels in response to stimulus;
applying a binary brain mask to select a plurality of regions of interest, wherein the binary brain mask is defined based on a group-level analysis of respective T-maps generated for the training subjects; and
applying a statistically-based feature selection procedure to the voxels of the T-map that are in the regions of interest, wherein the statistically-based feature selection procedure includes one or more of feature selection based on Pearson correlation, L1 regularization penalty feature selection, or principal component analysis.

21. The system of claim 13 wherein the classifier is one of:
a support vector machine classifier;
a Bayesian classifier;
a Gaussian Naïve Bayes classifier; or
a random forest classifier.

22. The system of claim 13 further comprising:
obtaining demographic data for the test subject,
wherein the input data set further includes the demographic data.

23. The system of claim 13 wherein the neuroimaging data collected from the test subject further includes anatomical neuroimaging data.

24. A computer-readable storage medium having stored therein program code instructions that, when executed by a processor in a computer system, cause the processor to perform a method comprising:
obtaining neuroimaging data from a test subject, the neuroimaging data including one or more of:
active-state functional neuroimaging data collected while the test subject performs a naturalistic language-based task;
resting-state functional neuroimaging data collected while the test subject is at rest; or
anatomical neuroimaging data characterizing one or more brain structures of the test subject;
extracting a feature set from the functional neuroimaging data;
defining an input data set that includes at least the feature set; and
determining a predicted NCD status for the test subject by analyzing the input data set using a classifier that has been trained using machine learning to predict the NCD status for an individual, wherein training of the classifier is based on corresponding input data sets obtained for a plurality of training subjects having known NCD status based on a neurocognitive assessment.

25. The computer-readable storage medium of claim 24 wherein the predicted NCD status is one of:
a binary status that distinguishes between a normal neurocognitive status and presence of NCD;
a continuous variable that represents severity of NCD; or
a continuous variable that represents likelihood of NCD.

26. The computer-readable storage medium of claim 24 wherein the predicted NCD status corresponds to one or both of a present condition of the test subject or a forecast of a future condition of the test subject.

27. The computer-readable storage medium of claim 24 wherein the neuroimaging data includes the active-state functional neuroimaging data and the active-state functional neuroimaging data is obtained using one or more of:
functional magnetic resonance imaging (fMRI) data;
functional near-infrared spectroscopy (fNIRS) data; or
magnetoencephalography (MEG) data.

28. The computer-readable storage medium of claim 27 wherein the naturalistic language-based task includes one or more of:
a movie-watching task in which the test subject is shown a movie clip that includes at least one segment with dialog or monolog and at least one non-speaking segment; or
a listening task in which the test subject is presented with a stream of spoken-language stimuli.
